(19) United States
(12) Patent Application Publication
(10) Pub. No.: US 2003/0125670 A1
(43) Pub. Date: Jul. 3, 2003

(54) Relating to a medicament cartridge

(75) Inventors: Christopher Nigel Langley, Warwickshire (GB); Shane Alistair Day, Warwick (GB); Paul William Gardner, Northampton (GB); Brian Charles Molyneux, Warwickshire (GB)

Correspondence Address: OLIFF & BERRIDGE, PLC, P.O. BOX 19928, ALEXANDRIA, VA, 22320, US

(73) Assignee: DCA Design International Limited, Warwick (GB)

(21) Appl. No.: 10/308,145

(22) Filed: Dec. 3, 2002

(30) Foreign Application Priority Data
Dec. 6, 2001 (GB) 0129173.1

(51) Int. Cl. A61M 5/00

(52) U.S. Cl. 604/232

(57) ABSTRACT

Many of those having diabetes take a combination of slow and fast acting types of insulin. It is important that different forms of medicament do not become confused and that the patient does not receive an incorrect medicament. In order to meet the appropriate standard, certain dimensional restrictions are placed upon the cartridge. A medicament cartridge that meets the requirements of this standard and permits more ready discrimination between medicament cartridges having different contents is provided. The medicament cartridge of the present invention is provided with an electrical element having specified electrical properties located on an external face of a displaceable plunger.

### FIELD OF THE INVENTION

The present invention relates to improvements in and relating to medicament cartridges for use with medication delivery apparatus and in particular but not exclusively for use with medicament delivery apparatus such as injection pens and infusers.

### BACKGROUND OF THE INVENTION

Medicament cartridges are known which code a cylinder having a bottleneck at a first end, a fluid impermeable membrane secured across the first end and a displaceable plunger located internally of the cylinder towards a second end of the cylinder. A medicament is stored in a chamber defined between the displaceable plunger, the membrane and a region of the cylinder therebetween. Such cartridges are used to store a variety of different medicaments such as insulin for diabetics or human growth hormone where a patient is entrusted with the self administration of an appropriate medicament regimen.

It is known that a person providing themselves, or others, with a medicament regimen may require more than one medicament as part of that regimen. For example many of those having diabetes take a combination of slow and fast acting types of insulin. It is important that the different forms of medicament do not become confused and that the patient does not receive an incorrect medicament.

### SUMMARY OF THE INVENTION

It is a problem that in order to meet the appropriate standard, ISO/FDIS 11608 Part 3, certain dimensional restrictions are placed upon the cartridge. It is an advantage of the present invention in that it provides for a medicament cartridge that meets the requirements of this standard and also enables a user, or alternatively, or additionally, a medicament dispensing apparatus into which a medicament cartridge in accordance with the present invention has been located, to discriminate more readily between medicament cartridges having different contents.

According to a first aspect of the present invention, a medicament cartridge comprising a cylinder having a bottleneck at a fist end, a fluid impermeable membrane secured across the first end and a displaceable plunger located internally of the cylinder towards a second end of the cylinder, a chamber defined by the displaceable plunger, the membrane and a region of the cylinder therebetween in which is stored a medicament to be dispensed, is provided with an electrical element having specified electrical properties located on an external face of the displaceable plunger.

This has as an advantage that the electrical element can be affixed to a standard cartridge at little addition cost. The electrical element may be visually configured to give an indication of the medicament within the medicament cartridge. The electrical properties of the electrical element may be configured to give an indication of the medicament within the medicament cartridge.

Preferably, the electrical element takes the form of a conductive disc.

Alternatively, the electrical element can take the form of two conductive rings joined by a resistive pad.

According to a second aspect of the invention a medication delivery device is provided with electrical contacts for contacting the electrical element of the first aspect of the invention.

Preferably, the medication delivery apparatus further includes a processor, the processor being able to recognise tat a contact between the electrical element and the electrical contacts of the Medication delivery device has been made on the basis of a signal received therefrom. Since the signal will be dependent upon the electrical properties of the electrical element, the processor be able to determine, for example from a look-up table, the nature of the medicament contained within the medicament cartridge.

Preferably, the medication delivery apparatus further comprises a display, the processor causing the display to show information relating to the medicament cartridge within the medication delivery apparatus dependant upon the nature of the signal received.

Preferably, the medication delivery apparatus further comprises a drive mechanism actuable by the processor, the processor only causing the drive mechanism to actuate when a recognised signal is received from the contacts. Thus, in the absence of a cartridge in accordance with the first aspect of the present invention the drive mechanism of the medication delivery apparatus will not be actuated.

### BRIEF DESCRIPTION OF THE DRAWINGS

The invention will now be described, by way of example only, with reference to the accompanying drawings in which:

FIG. 1 shows details of a known medicament cartridge;

FIG. 2 shows a medicament cartridge in accordance with the first aspect of the invention;

FIG. 3 shows schematically a pat of a medication delivery apparatus for use with a medicament cartridge in accordance with the first sect of the invention;

FIG. 4 shows schematically a part of a medication delivery apparatus for use with a medicament cartridge in accordance with the first aspect of the invention; and

FIG. 5 shows a second medicament cartridge in accordance with the first aspect of the invention.

### DETAILED DESCRIPTION OF EMBODIMENTS

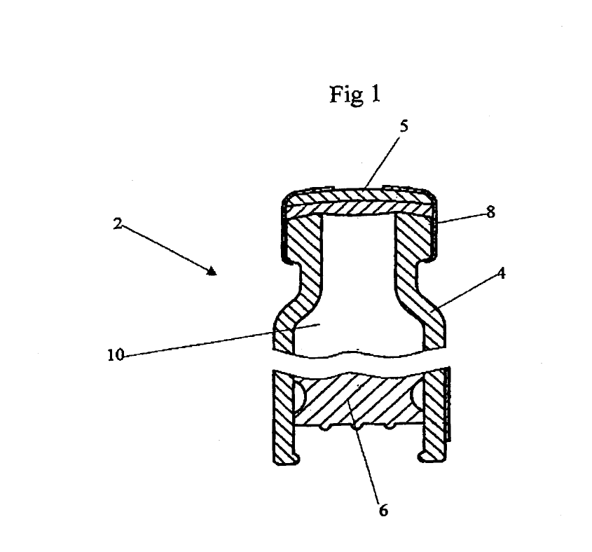

Referring first to FIG. 1, there is shown a known medicament cartridge 2. The medicament cartridge 2 comprises a cylinder 4 having a bottleneck at a first end, together with a fluid impermeable membrane 5 and a displaceable plunger 6. The fluid impermeable membrane 5 is secured in position by a cap 8 that is beaded about a flange 7. The displaceable plunger 6 is located internally of the cylinder 4 towards a second end of the cylinder 4. A chamber 10 is defined between the displaceable plunger 6, the membrane 5 and a region of the cylinder 4 therebetween. A medicament to be dispensed is stored within the chamber 10.

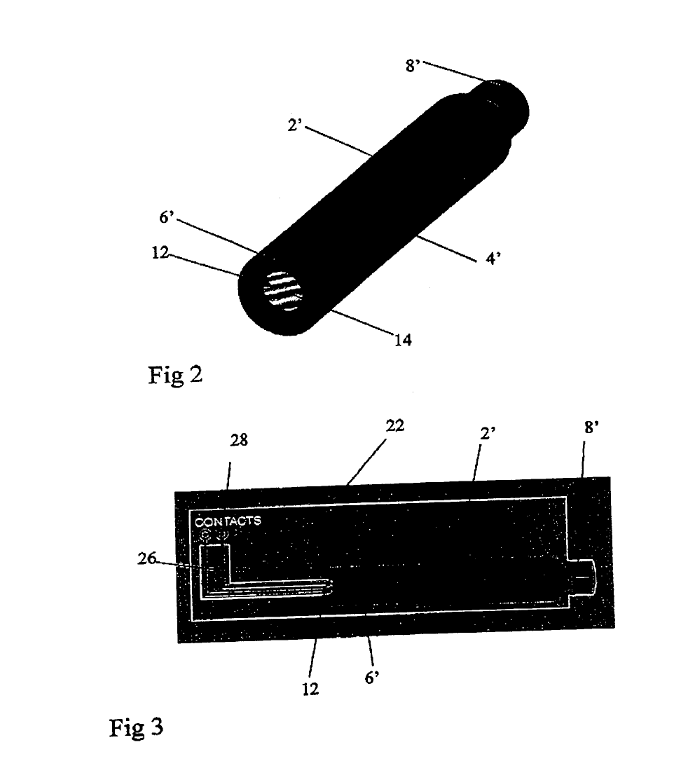

Referring now to FIG. 2, there is shown a first embodiment of a medicament cartridge modified in accordance with the present invention. An electrical element 12 is located on an external face of a displaceable plunger 6′. In the embodiment of FIG. 2, the electrical element comprises a conductive disc 14 having predetermined electrical properties.

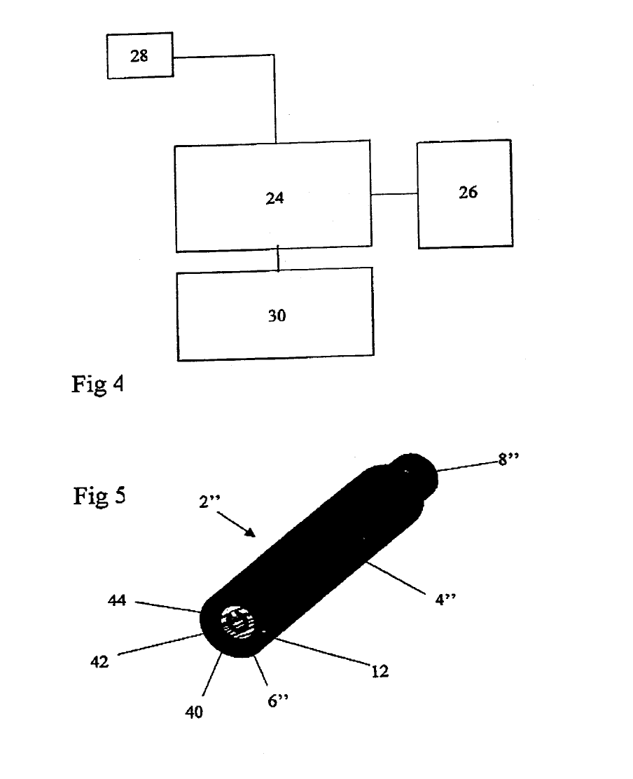

The medicament cartridge may be used with a medicament delivery apparatus 20 such as that illustrated in outline in FIG. 3 and shown schematically in FIG. 4. The medicament delivery apparatus comprises a housing 22 having a region within which a medicament cartridge 2′ may be retained. The housing 22 flyer includes a processor 24 and a drive mechanism 26. The drive mechanism 26 is actuable by the processor 24 to drive the displaceable plunger 6′ within the cylinder 2′ towards a cap 8′. In use, the cap 8′ is provided with a needle unit having a needle that pierces the fluid impermeable membrane to allow the egress of medicament when the volume of the chamber 10′ is decreased.

The housing 22 additionally includes contacts 28 between the processor 24 and the region within which the medicament cartridge 2′ is retained. In use, the processor seeks to send a signal through the contacts 28.

If no medicament cartridge in accordance with the present invention is present, the signal will not pass. The processor 24 will detect the absence of a returned signal. If the processor 25 has been programmed to operate only in the presence of a medicament cartridge in accordance with the present invention the processor 24 will not then actuate the drive mechanism 26.

If a medicament cartridge in accordance with the present invention is present, a signal will be returned dependant upon the electrical properties of the electrical element. If the processor has been programmed to operate only in the presence of a medicament cartridge in accordance with the present invention, the processor 24 will permit the drive mechanism 26 to be actuated.

Further, since the returned signal is dependant upon the electrical properties of the electrical element 12, the predetermined properties of the electrical element 12 can be chosen to provide an indication of the medicament stored within the medicament cartridge to which the electrical element 12 has been affixed. The processor 24 may be provided with a look-up table in which certain values corresponding to possible returned values have been stored. If the medicament dispensing apparatus is further provided with a display 30, the processor 24 may use the display to provide an indication regarding the contents of the retained medicament cartridge to a user.

A second embodiment of the present invention is shown in FIG. 5. The medicament cartridge is substantially identical to that of FIG. 2 and like reference numerals have been used to refer to like parts. In this embodiment, the electrical element 12 comprises a first conductive ring 40, a second conductive ring 42, and a resistive pad 44 joining the first conductive ring and the second conductive ring. Contacts are provided within a medicament delivery apparatus as shown for example in FIG. 4 such that the contacts are disposed across the first and second conductive rigs 40,42 to measure the resistance of the resistive pad 44.

1. A medicament cartridge comprising a cylinder having a bottleneck at a first end, a fluid impermeable membrane secured across the first end and a displaceable plunger located internally of the cylinder towards a second end of the cylinder, a chamber defined by the displaceable plunger, the membrane and a region of the cylinder therebetween in which is stored a medicament to be dispensed, wherein the medicament cartridge is further provided with an electrical element having specified electrical properties located on an external face of tie displaceable plunger.

2. A medicament cartridge according to claim 1, wherein the electrical element takes the form of a conductive disc.

3. A medicament cartridge according to claim 1, wherein the electrical element takes the form of two conductive rings joined by a resistive pad

4. A medication delivery device wherein the medication delivery device is provided with electrical contacts for contacting the electrical element of a medicament cartridge according to any previous claim.

5. A medication delivery device according to claim 4, wherein the medication delivery apparatus further includes a processor, the processor being able to recognise that a contact between the electrical element and the electrical contacts of the medication delivery device has been made on the basis of a signal received therefrom.

6. A medication delivery device according to claim 5, wherein the medication delivery apparatus further comprises a display, the processor causing the display to show information relating to the medicament cartridge within the medication delivery apparatus dependant upon the nature of the signal received.

7. A medication delivery device according to claim 4, wherein the medication delivery apparatus further comprises a drive mechanism actuable by the processor, the processor only causing the drive mechanism to actuate when a recognised signal is received from the contacts.

8. A medication delivery device according to either of claim 5, wherein the medication delivery apparatus further comprises a drive mechanism actuable by the processor, the processor only causing the drive mechanism to actuate when a recognised signal is received from the contacts.
